(19) United States
(12) Patent Application Publication
(10) Pub. No.: US 2023/0201633 A1
(43) Pub. Date: Jun. 29, 2023

(54) Clothing Element for Protection from Organic Structures, System and Method

(71) Applicants: Carolin-Sophie Schnölzer, Bad Oeynhausen (DE); Leonie-Marie Schnölzer, Bad Oeynhausen (DE)

(72) Inventors: Carolin-Sophie Schnölzer, Bad Oeynhausen (DE); Leonie-Marie Schnölzer, Bad Oeynhausen (DE)

(21) Appl. No.: 17/920,277

(22) PCT Filed: Apr. 20, 2021
(86) PCT No.: PCT/EP2021/060248

§ 371 Date: Oct. 20, 2022

(30) Foreign Application Priority Data
Apr. 20, 2020 (DE) 10 2020 110 699.8

(51) Int. Cl. A62B 17/00; A61L 9/20

(52) U.S. Cl. CPC A62B 17/006; A61L 9/20; A61L 2209/111; A61L 2209/12

(57) ABSTRACT

The invention relates to a clothing element (10) comprising a wearing portion (11) for wearing the clothing element (10) in a wearing position (I) by a wearer (1), and an emission unit (12) for emitting electromagnetic radiation (200) for killing organic structures (2), in particular in the form of pathogens and/or insects. Furthermore, the invention relates to a system (5) comprising a clothing element (10) and an external apparatus (4), and to a method (100) for protecting a body region (202) of a wearer (1).

The invention relates to a clothing element, a system comprising a clothing element and an external apparatus, and a method for protecting a body region of a wearer.

To protect against pathogens, it is common practice, particularly in the medical field, to use masks that cover the mouth and nose. This is intended to prevent the absorption of body fluids from the near ambient air and/or the release of body fluids into the near ambient air in order to prevent the spread of pathogens. However, the masks sometimes offer only insufficient protection, since, for example, the eyes of the wearer remain uncovered and are thus exposed to possible pathogens in the air. Furthermore, many people have the habit of regularly touching their faces, so that pathogens picked up with the hands can reach the mucous membranes and thus gain access to the body of the wearer.

Furthermore, especially in times when larger parts of the population are affected by infectious pathogens such as influenza or corona viruses, protective measures that otherwise only apply to medical personnel must also be applicable in everyday life. However, such masks are only suitable for everyday use to a limited extent, as they can quickly slip out of place. The fact that the masks cover part of the wearer's face opaquely is also impractical and unfashionable for many areas of everyday life.

It is an object of the present invention to at least partially overcome the aforementioned disadvantages known from the prior art. In particular, it is a task of the present invention to enable improved protection of a wearer from organic structures, such as pathogens and/or insects, by means of a clothing element which is preferably suitable for everyday use.

The foregoing problem is solved by a clothing element having the features of claim 1, a system having the features of claim 15, and a method having the features of claim 16. Further features and details of the invention result from the respective subclaims, the description and the drawings. In this context, features and details described in connection with the clothing element according to the invention also apply, of course, in connection with the system according to the invention and/or the method according to the invention, and vice versa in each case, so that reference is or can always be made mutually with regard to the disclosure concerning the individual aspects of the invention.

In accordance with a first aspect of the invention, there is provided a clothing element. The clothing element comprises a wearing portion for wearing the clothing element in a wearing position by a wearer. Further, the clothing element comprises an emission unit for emitting, i.e. in particular for emitting, an electromagnetic radiation for killing organic structures, in particular in the form of pathogens and/or insects. In the wearing position, the electromagnetic radiation of the emission unit can generate a protected area by which a body region of the wearer is at least partially or completely protected from the organic structures.

In particular, the clothing element may also be referred to as a garment. For example, the clothing element may be a pair of glasses or a headgear, preferably in the form of a helmet, a peaked cap or a hat. It is further conceivable that the clothing element is a fashion item, an accessory, in particular a wearable, and/or part of a work outfit. It may be envisaged that the clothing element is attachable to a helmet or the like, in particular in the manner of a headlamp.

In particular, the wearing portion is configured to allow the clothing element to be worn as intended by the wearer in the wearing position. The wearer may be, for example, a person wearing the clothing element. Preferably, the wearing portion may comprise a textile, a rubber band and/or a plastic. In the wearing position, the wearing portion may be arrangeable indirectly or directly on the body, in particular on the head, of the wearer. For example, the wearing portion may be configured for attachment to a further part of a clothing of the wearer.

The organic structures can be understood in particular as pathogens, such as viruses or bacteria, and/or insects. In particular, the organic structures which can be killed by the electromagnetic radiation are foreign organic structures, i.e. organic structures which are foreign to the body of the wearer. Killing the organic structures may be understood in particular as rendering the organic structures harmless to the body of the wearer. For example, killing may involve rendering bacteria harmless and/or deactivating viruses. In particular, the organic structures can be at least partially damaged and/or reduced by the electromagnetic radiation in such a way that they do not endanger the immune system of the wearer or endanger it to a lesser extent. For protection against insects, the electromagnetic radiation may comprise, for example, a laser, in particular of low intensity, so as not to allow the insects to penetrate to the body region. Thus, effective protection against mosquitoes may be producible.

The emission unit may in particular be designed/configured to emit the electromagnetic radiation in the form of light. For generating the protected area, the emission unit may comprise a reflector which focuses the electromagnetic radiation, in particular in an advantageous radiation direction. By means of the protected area, a protective barrier is thus generated in particular which can prevent the organic structures from advancing to the body region in a harmful manner. It is further conceivable that the emission of the radiation is line-like, area-like and/or in at least one beam. If droplets containing the organic structures are emitted into the air by bystanders, for example by coughing, sneezing or speaking, these droplets pass through the protected area so that the organic structures are at least partially killed. It is further conceivable that the emission unit comprises an indicator light for indicating an emission of the radiation. Thus, it may be visible to the outside that the emission of the radiation is taking place, in particular at this moment.

The electromagnetic radiation may be selected in an area that is not visible to a human, such that the body region is still visible to a opposite person of the wearer. For example, if the body region is the face of the wearer, an opposite person can still fully see the facial expressions of the wearer. In particular, the protected area may be invisible so that even in an extreme situation, such as a pandemic, a high degree of normality can be maintained, at least visually, and/or visual impairment can be reduced or avoided. Furthermore, the clothing element may be integrated into an everyday outfit of the wearer, in particular fashionably, so that it is readily worn by the wearer, which may for example increase the willingness of large parts of the population to take protective measures. At the same time, the importance of regular disinfection of the clothing element itself may be reduced or unnecessary, since protection against the organic structures is provided via the electromagnetic radiation, in particular without contact. Furthermore, for example, a hand or an object which the wearer guides to the body region can also be disinfected by the electromagnetic radiation, at least in some areas. Furthermore, the wearer is preferably not disturbed by the clothing element when eating or drinking.

In the context of the invention, it is further conceivable that the radiation emittable by the emission unit comprises a UV radiation, preferably a UV-C radiation, more preferably a Far-UV-C radiation. In particular, it has been recognized in the context of the invention that UV radiation, preferably in the form of UV-C radiation, can have a damaging effect on pathogens, in particular on viruses. For example, influenza and/or Sars viruses, in particular Sars-CoV-2 viruses, may thereby be harmful, so that they do not cause disease in the wearer of the clothing element. The Far UV-C radiation, in particular also designable/configurable as Far UVC or Far UV-C radiation, may be highly efficient, in particular against pathogens. In particular, the UV radiation may comprise or consist of a wavelength between 100 nm and 400 nm, preferably between 100 nm and 280 nm, more preferably of 254 nm, 222 nm or 185 nm. In particular, the electromagnetic radiation may be monochromatic. For example, a wavelength of 254 nm has the advantage of being harmful to a broad spectrum of organic structures, so that different types of the organic structures can be killed. A wavelength of 222 nm has the advantage that such radiation is absorbable by a stratum corneum of human skin or human eye, so that a risk of damage to the wearer is reduced or avoided. A wavelength of 185 nm has also been found to be beneficial in combating viruses. In particular, an intensity or dose of radiation at about 2 mJ/cm² or 200 J/m² to 300 J/m² may be provided and sufficient. Advantageously, for filtering and/or generating the wavelengths, the emission unit may comprise a quartz glass. Preferably, the emission unit comprises a krypton chlorine excimer lamp for generating the electromagnetic radiation, in particular with a wavelength of 222 nm. For example, at a wavelength of 222 nm and a dose of 2 mJ/cm², more than 95% of H1N1 viruses can be deactivated in an air region.

Preferably, in a clothing element according to the invention, it may be provided that the emission unit comprises at least one emission element in the form of an LED, a laser and/or an excimer element. The LED may enable an energy-efficient operation of the emission unit, whereby emission intervals may be extended and/or a mobility of the clothing element may be increased. In particular, the LED may be an LED for emitting UV light. Preferably, the emission unit may comprise a plurality of emission elements, in particular a plurality of LEDs. The LEDs may, for example, be arranged in a matrix and/or row-like manner to define the protected area. In particular, the LEDs may form an LED matrix and/or an LED array. In this context, the LEDs may preferably be connected in one or more groups, for example in series or in parallel. Due to a compact design/configuration, the at least one emission element can advantageously be integrated into a clothing of the wearer. A laser has the advantage that it enables a particularly targeted and/or concentrated emission of the electromagnetic radiation. An excimer element, which may in particular be embodied as an excimer lamp and/or excimer laser, may preferably have a KrCl filling, i.e. a krypton chlorine filling. This has the advantage that natively focused electromagnetic radiation with a wavelength of 222 nm can be emitted in a simple manner. This may enhance the killing of the organic structures.

It is further conceivable, in a clothing element according to the invention, that the electromagnetic radiation in the wearing position has a radiation direction by which contact of the electromagnetic radiation on the body region is prevented. If it is ensured that the wearer is not irradiated, for example a stronger intensity can be used for the radiation. Preferably, the emission unit comprises a focus element for directing the radiation in the radiation direction. For example, the focus element may comprise a reflector, a lens or the like. It is conceivable that the focal element comprises one or more mirror surfaces to provide the radiation direction and/or to focus the radiation. To prevent the radiation from contacting the body region, the radiation direction may be directed parallel to the body region and/or away from the body region. By specifying the radiation direction, the emission of the radiation may be definable in a simple manner with respect to the clothing element, the wearer and/or the environment of the wearer. Additionally or alternatively, it may be provided that an intensity of the radiation is such that the radiation does not reach the body region or a further body region of the wearer, or reaches the wearer only in an attenuated form.

It is further conceivable in a clothing element according to the invention that the emission unit is designed/configured to emit the radiation in a cone-like shape, in particular with an opening angle of the cone-like radiation of 20° to 40°, preferably of 30°. In this way, the protected area can be given a cone-like shape. The opening angle of the cone-like radiation can in particular be understood as an angle between two lateral surfaces and/or between two lateral lines of the cone-like radiation. In this way, a sufficiently large protected area can be created so that organic structures flying through the protected area can be supplied with an amount of radiation which leads to at least partial killing of the organic structures. In particular, the cone-like shape of the protected area can simultaneously direct the radiation in such a way that the radiation does not strike the body region.

Within the scope of the invention, it is further conceivable that the body region protected at least in part by the protected area is a facial region of the wearer. The facial area may comprise the full face of the wearer or merely areas of the wearer's face that are sensitive to pathogens, such as the mouth, nose and/or eyes. In particular, when the organic structures are pathogens transmitted by droplet infection, this may provide a high level of protection for the wearer. Advantageously, to protect the facial region, the radiation may be emitted at a distance from the facial region.

Within the scope of the invention, it is further conceivable that the wearing portion is configured for wearing the clothing element on the head of the wearer, preferably wherein a shield section extends from the wearing portion for shielding the body region on which the emission unit is arranged. Thereby, in an advantageous manner, the face area of the wearer may be protectable by the protected area. The shield section may form, for example, a shield of a cap, in particular a baseball cap, or a brim of a hat. Preferably, the shield section may comprise a reinforcing member made of plastic or cardboard to stabilize the shield section. In particular, the shield section may simultaneously provide sun protection for the face area of the wearer, so that a multiple benefit may be achieved. Furthermore, the emission unit may be arranged at a distance from the body of the wearer due to the arrangement on the shield section and, in particular, may simultaneously embody an everyday fashion. Further, the distance may improve a response capability, as the organic structures may travel a greater distance on their way to the body region and thus be exposed to radiation for a longer time, or a detection time for detecting a hazard potential of the organic structures may be extended. The arrangement at the shield section may comprise an arrangement of the emission unit externally at the shield section or an integration of the emission unit into the shield section. For example, the emission unit may be at least partially sewn into the shield section if the shield section comprises a textile or textile cover.

Furthermore, in a clothing element according to the invention, it may advantageously be provided that an energy supply unit is provided for providing electrical energy for the emission unit, in particular wherein the energy supply unit comprises a connecting element for connecting to an external energy source and/or an internal energy storage, in particular in the form of a battery. The external energy source may comprise, for example, a socket connected to a mains supply. To this end, the connection element may comprise a plug connector and/or a cable. For example, the connection element may comprise a USB port for connecting to the external energy source. The external energy source may be used to charge the internal energy storage device and/or operate the emission unit. In particular, for wearers performing an activity in a fixed location, such as bus drivers, cashiers or the like, a permanent power connection of the clothing element may be advantageous. This may eliminate the need to change the clothing element during the activity, while ensuring permanent operation and thus protection of the wearer. Furthermore, due to the internal energy storage, a mobility of the clothing element and thus of the wearer may be increased. As a result, the emission unit may be operable independently of an external power source (external energy source). In particular, the battery may be a rechargeable battery. For example, the battery may be recharged overnight so that the clothing element can be used during the day.

Furthermore, in a clothing element according to the invention, it may advantageously be provided that an internal control unit comprising an emission module for controlling the emission of the emission unit is provided. The emission and/or the intensity of the electromagnetic radiation may be controllable continuously or in a pulse-like manner, in particular at regular or irregular intervals. The control unit may, for example, implement a manual control command from the wearer in order to control the emission unit. Thus, it is conceivable that the clothing element comprises a control element for actuating the emission unit by the wearer. The operating element may be in communication with the control unit. Preferably, the control unit comprises a processor and/or a microprocessor. In particular, the control unit may comprise a microcontroller to control the emission unit. The emission module may be implemented in hardware and/or software in the control unit. Preferably, the control unit is arranged at the shield section and/or at the wearing portion.

Furthermore, in a clothing element according to the invention, it is conceivable that the control unit is connected to a sensor unit, preferably an acoustic and/or optical sensor unit, for acquiring environmental data in the form of sensor data, in particular wherein the emission module is designed/configured to control the emission of the electromagnetic radiation of the emission unit dependent on the sensor data. The environmental data may in particular comprise data of an environment of the wearer. Preferably, the sensor unit may be arranged at the shield section and/or at the wearing portion. Through the detection of optical and/or acoustic sensor data, it may be possible to determine, for example, whether there is an increased risk situation for the wearer starting from the environment. Thus, it can be determined whether there is sneezing or coughing in the environment of the wearer or by the wearer himself, in order to emit the electromagnetic radiation by the emission unit thereupon. The control of the emission unit in dependence on the sensor data may in particular comprise switching the emission unit on and/or off and/or changing an emission interval of the radiation and/or an intensity of the radiation.

Furthermore, in a clothing element according to the invention, it can advantageously be provided that the control unit is connected to a position unit for detecting environmental data in the form of position data of a geographical position of the clothing element, in particular wherein the emission module is designed/configured for controlling the emission of the electromagnetic radiation of the emission unit dependent on the position data. Preferably, the position unit may be arranged at the shield section and/or at the wearing portion. The position data may comprise, for example, satellite data in the form of GPS data or Galileo data to infer the geographical position of the clothing element and thus the wearer. This can be used, for example, to determine whether the wearer is in a risk area for a disease or a particular type of insect. The control of the emission unit in dependence on the position data may in particular comprise switching the emission unit on and/or off and/or changing an emission interval of the radiation and/or an intensity of the radiation.

Furthermore, in a clothing element according to the invention, it may advantageously be provided that the control unit is connected to a communication unit for receiving environmental data in the form of external data from an external apparatus, in particular wherein the emission module is configured for controlling the emission of the electromagnetic radiation of the emission unit in dependence on the external data. Preferably, the communication unit may be arranged at the shield section and/or at the wearing portion. Preferably, the communication unit is adapted to transmit the environmental data in the form of the external data, the position data and/or the sensor data. Thus, in particular, the communication unit may be configured for bidirectional communication with the external apparatus or for multidirectional communication with a plurality of external apparatuses. The external apparatus may comprise, for example, a mobile terminal, such as a smartphone. Furthermore, it is conceivable that the external apparatus comprises another clothing element of another wearer or a central control unit, such as a server. The communication unit may comprise a WLAN interface, a Bluetooth interface, a cellular interface, such as a GSM interface, and/or the like for receiving and/or transmitting the environmental data. The external data may enable the clothing to be informed whether an increased risk situation arises from the environment of the wearer, for example due to a presence of an infected person. The external data may comprise assessed data or raw data, such as sensor data. Controlling the emission unit in dependence on the external data may in particular comprise switching the emission unit on and/or off and/or changing an emission interval of the radiation and/or an intensity of the radiation. Furthermore, it is conceivable that a software of the control unit can be updated via the communication unit.

Furthermore, in a clothing element according to the invention, it can advantageously be provided that the control unit comprises an evaluation module for producing an evaluation of a hazard potential of penetration of organic structures to the wearer dependent on the environmental data, in particular dependent on the sensor data, the position data and/or the external data, in particular wherein the emission module is designed/configured for controlling the emission of the electromagnetic radiation of the emission unit dependent on the evaluation. For the evaluation, the sensor data, the position data and/or the external data can be combined. In this context, the hazard potential may be determinable on the basis of reference data. By evaluating the hazard potential, the emission of the radiation can preferably be controlled, in particular triggered, on an event-based basis. For example, it can be taken into account whether a person infected with a pathogen is located in the vicinity of the wearer, whether a person in the vicinity of the wearer sneezes, whether the wearer is located in a risk area or the like. Thus, it is further conceivable that a larger gathering of people, such as in a subway, or an unfavorable wind direction directed towards the wearer, is detected and/or classified as a potential hazard. In particular, the evaluation may be based on a knowledge database comprising the reference data. The knowledge database may be integrated into the evaluation module and/or the external apparatus. Preferably, the evaluation module is designed/configured to be teachable. In particular, the evaluation may be improvable by machine learning in the course of an operation of the clothing element. For this purpose, the knowledge base may be expandable based on the acquired environmental data. Preferably, the control unit and/or the external apparatus comprises an artificial intelligence to adapt the evaluation, in particular in dependence on the sensor data. Advantageously, the evaluation may comprise a classification of the hazard potential in order to enable an improved control of the emission unit.

Furthermore, in a clothing element according to the invention, it may advantageously be provided that a plurality of emission units are provided for emitting an electromagnetic radiation for killing organic structures, in particular wherein the emission units have different, preferably predetermined and/or intrinsic, radiation parameters and/or wherein the emission units are arranged successively in such a way that the body region in the wearing position can be at least partially or completely protected by each of the emission units. Advantageously, the emission units may be controllable independently of each other and/or jointly. The radiation parameters may comprise, for example, a radiation intensity, a wavelength of the emitted radiation, a radiation dose and/or the like. It is conceivable that one of the emission units is determinable by the control unit in dependence on the radiation parameter of the respective emission unit and in dependence on the sensor data, the external data and/or the position data for generating the protected area. Furthermore, a target parameter may be taken into account when determining the emission unit. For example, it may be provided that a radiation dose as low as possible is selected in order to increase the safety of the clothing element, in particular with respect to side effects of the electromagnetic radiation. Thus, one of the emission units may be selectable whose radiation parameter is advantageous for the particular environmental situation. The sequential arrangement may ensure that each of the emission units provides protection for the body region. For example, this may provide a plurality of protective screens arranged in front of each other. Furthermore, it is conceivable that the remaining or some of the remaining emission units can be switched off or are switched off by the control unit while the emission unit selected in dependence on the ambient situation is operated. Preferably, the emission units are arranged in succession in the wearing position perpendicular or substantially perpendicular to the body region, for example along a field of view of the wearer. As a result, a protected area can advantageously be generated by each of the emission units. According to another aspect of the invention, a system is provided. The system comprises at least one clothing element, in particular a clothing element according to the invention, and an external apparatus for data communication with the clothing element. The clothing element has a wearing portion for wearing the clothing element in a wearing position by a wearer. Further, the clothing element has an emission unit for emitting an electromagnetic radiation for killing organic structures, in particular in the form of pathogens and/or insects. In the wearing position, the electromagnetic radiation of the emission unit can generate a protected area by which a body region of the wearer is at least partially protected from the organic structures. The clothing element further comprises a communication unit for receiving environmental data in the form of external data from the external apparatus. Furthermore, an evaluation module for generating an evaluation of a hazard potential of a penetration of organic structures to the wearer depending on the environmental data is provided. The clothing element further comprises an internal control unit having an emission module for controlling emission of electromagnetic radiation from the emission unit in response to the evaluation.

Thus, a system according to the invention brings the same advantages as have already been described in detail with reference to a clothing element according to the invention. Preferably, the evaluation module is integrated in the control unit of the clothing element. However, it is also conceivable that the evaluation module is integrated into the external apparatus. For example, the communication unit of the clothing element and the external apparatus may be configured to communicate bidirectionally with the external apparatus. In this way, for example, sensor data of the clothing element may be provided to the external apparatus and only the evaluation of the environmental data may be sent back to the clothing element. In this way, a computing capacity for evaluating the environmental data can be outsourced to the external apparatus, whereby the clothing element, in particular the control unit of the clothing element, can be of smaller design/configuration.

The system, and in particular the evaluation of the environmental data, can thus provide further improved protection of the wearer of the clothing element from potentially harmful organic structures by the clothing element.

According to a further aspect of the invention, there is provided a method of protecting a body region of a wearer of a clothing element, in particular a clothing element according to the invention, from organic structures while the clothing element is in a wearing position in which the clothing element is worn by the wearer. The method comprises the following steps/stages:
Detection of environmental data of an environment of the wearer, in particular by the clothing element,
Producing an evaluation of a hazard potential of penetration of organic structures to the wearer dependent on the environmental data, in particular by the clothing element,
creating a protected area in response to the evaluation by emitting electromagnetic radiation to kill the organic structures by the clothing member so that a body region of the wearer is at least partially protected by the protected area from advancement of the organic structures to the body region.

Thus, a method according to the invention brings the same advantages as have already been described in detail with reference to a clothing element and/or system according to the invention. Preferably, the clothing element is part of a system according to the invention. Acquiring the environmental data may comprise sensing and/or obtaining the environmental data. In particular, the environmental data may comprise sensor data, position data and/or external data from an external apparatus. Producing the evaluation may be performed by the clothing element, in particular by the evaluation module of the clothing element, and/or by the external apparatus. The method, and in particular the evaluation of the environmental data, can thus provide further improved protection of the wearer of the clothing element from possibly harmful organic structures by the clothing element.

Further advantages, features and details of the invention will be apparent from the following description, in which embodiments of the invention are described in detail with reference to the drawings. In this connection, the features mentioned in the claims and in the description may each be essential to the invention individually or in any combination. It schematically shows:

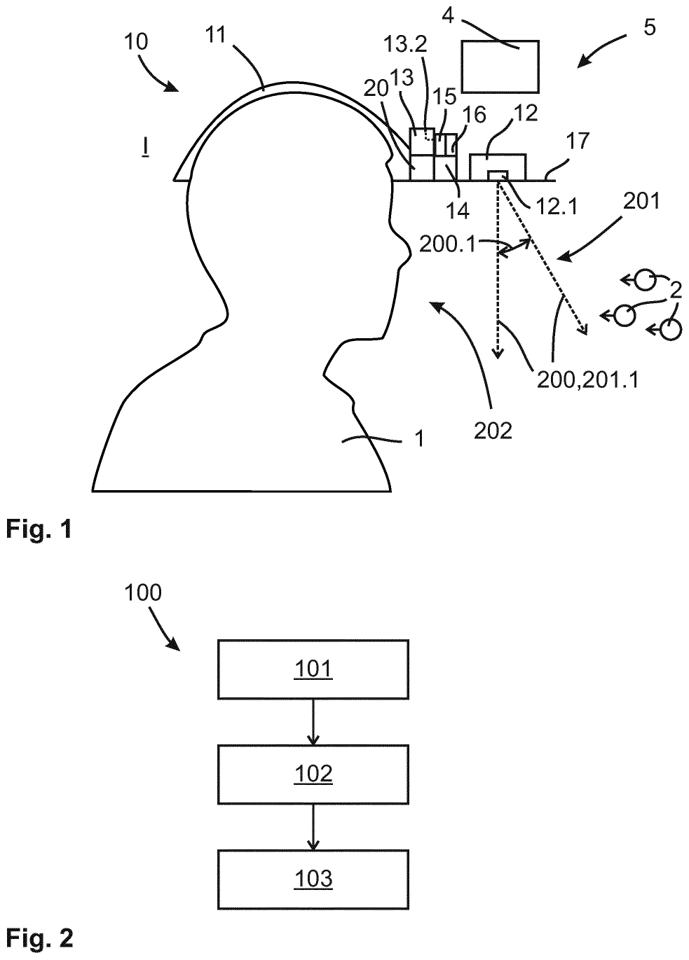

FIG. 1 a system according to the invention with a clothing element according to the invention in a first embodiment,

FIG. 2 a method according to the invention for protecting a body region of a wearer of the clothing element in schematic representation of method steps/stages,

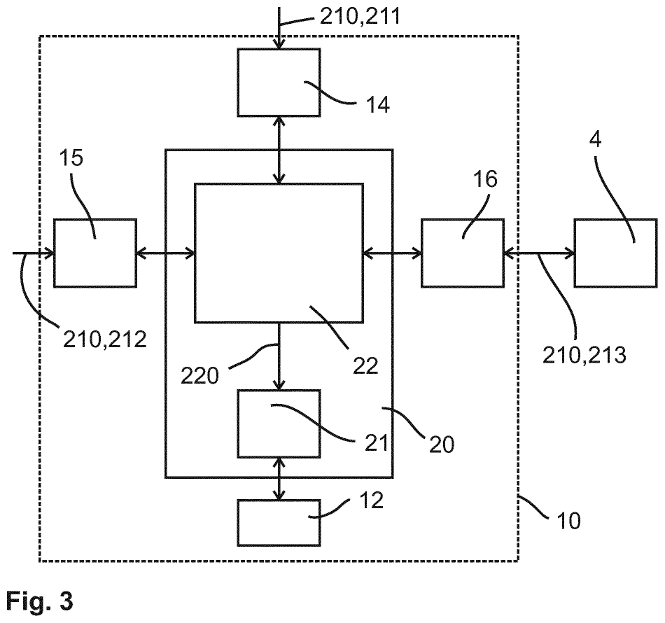

FIG. 3 an internal control unit of the clothing element,

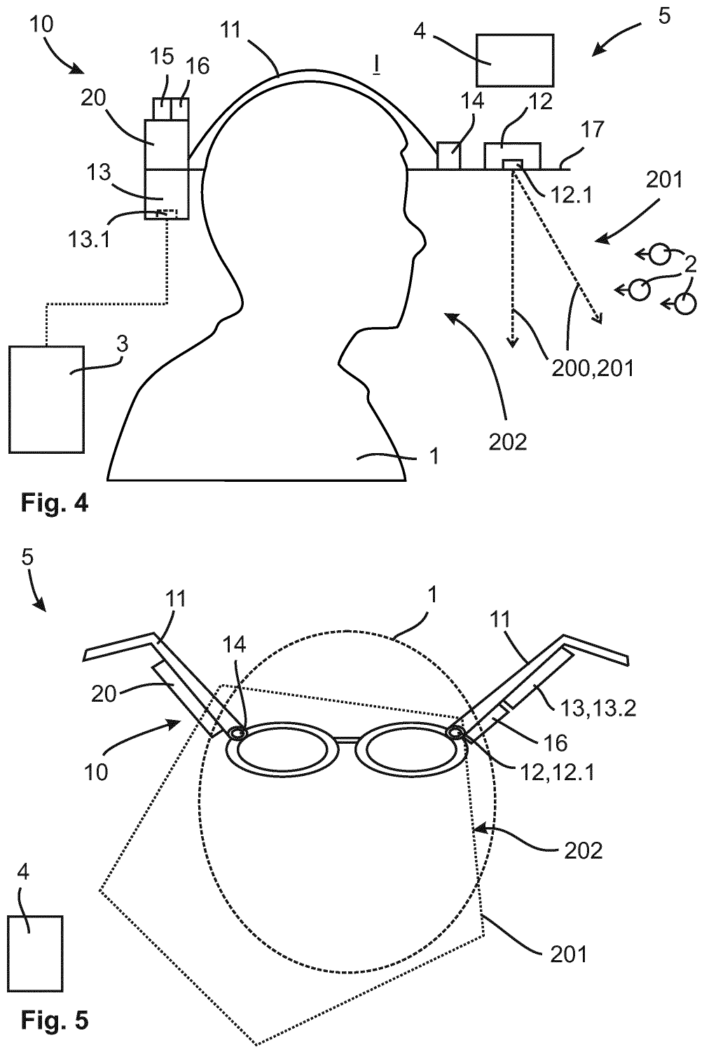

FIGS. 4-5 Systems according to the invention, each with a clothing element according to the invention in further examples of embodiment, and

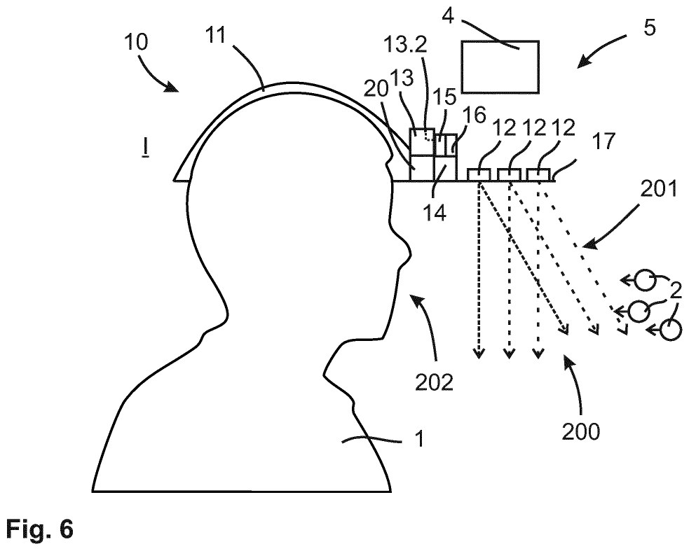

FIG. 6 a system according to the invention with a clothing element according to the invention in a further embodiment.

In the following description of some embodiments of the invention, the identical reference signs are used for the same technical features even in different embodiments.

FIG. 1 shows a system 5 according to the invention with a clothing element 10 according to the invention in a first embodiment. In this case, the clothing element 10 is designed/configured as a headgear of a wearer 1 and is located in a wearing position I in which the clothing element 10 is worn by the wearer 1. A method 100 according to the invention for protecting a body region 202 of the wearer 1 of the clothing element 10 is further illustrated in FIG. 2. In this case, the steps/stages of the method 100 are carried out while the clothing element 10 is in the wearing position I.

For wearing the clothing element 10 by the wearer 1, the clothing element 10 has a wearing portion 11 for wearing, in particular placing, the clothing element 10 in a wearing position I by the wearer 1. Extending from the wearing portion 11 in the front head region is a shield section 17, on which an emission unit 12 for emitting an electromagnetic radiation 200 for killing organic structures 2 is arranged. The organic structures 2 may be, for example, pathogens in the form of viruses and/or bacteria or insects. Thus, many pathogens are transmitted via the ambient air by a so-called droplet infection. A protected area 201 can therefore be generated by the electromagnetic radiation 200, by means of which the body region 202 of the wearer 1 is at least partially or completely protected from the organic structures 2. To generate the protected area 201, the electrical radiation 200 can be emitted continuously or at intervals, in particular in a pulse-like manner, by the emission unit 12, so that the organic structures 2 are exposed to the radiation 200 when the organic structures 2 advance towards the body region 202. In this way, any danger to the wearer 1 or the wearer's immune system that may be posed by the organic structures 2 can be reduced or avoided altogether. Here, the body region 202 is a facial region of the wearer 1 which includes the mouth, nose and eyes of the wearer 1. As a result, harmful penetration of pathogens into the mucous membranes of the wearer 1 can be reduced or avoided. In particular, the air supplied to the body region 202 may be disinfected by the radiation 200.

For this purpose, the emission unit 12 comprises at least one emission element 12.1 in the form of an LED, a laser and/or an excimer element. In particular, the emission element 12.1 is designed/configured for emitting the radiation 200 as a UV radiation, preferably a UV-C radiation, particularly preferably a Far-UV-C radiation. Thereby, the radiation 200 can be emitted in a cone-like manner, in particular with an opening angle 200.1 of 20° to 40°, preferably of about 30°. By the arrangement at the shield section 17, a radiation direction 201.1 of the radiation 200 can furthermore be predeterminable, which prevents the radiation 200 from contacting the body region 202 of the wearer 1. Thus, the wearer 1 itself may be protected from the radiation 200 and/or a higher intensity of the radiation 200 may be selected than when the wearer 1 itself is exposed to the radiation 200. For this purpose, the radiation 200 partly runs parallel to the body region 202 and partly runs away from the body region 202. Preferably, the emission unit 12 comprises a plurality of emission elements 12.1 which may in particular be arranged in the form of an LED matrix and/or an LED array.

In order to provide electrical energy for the operation of the emission unit 12, the clothing element 10 comprises an energy supply unit 13 comprising an internal energy storage 13.2. Preferably, the energy storage 13.2 is a rechargeable battery. This allows the clothing element 10 to be worn by the wearer 1 without the need for proximity to an external energy source 3.

Further, the clothing element 10 comprises an internal control unit 20 having an emission module 21 for driving the emission unit 12. The control unit 20 is shown in FIG. 3. For a detection 101 of environmental data 210 of an environment of the wearer 1, the control unit 20 is in communication with a sensor unit 14, a position unit 15 and a communication unit 16. Preferably, the control unit 20, the energy supply unit 13, the sensor unit 14, the position unit 15 and the communication unit 16 are arranged at the shield section 17. Through the sensor unit 14, environmental data 210 can be acquired in the form of sensor data 211. For example, an activity, such as sneezing or coughing, of other persons in the environment may be detected by the sensor unit 14. For this purpose, the sensor unit 14 may be configured to acoustically and/or visually detecting the sensor data 211. Environmental data 210 in the form of position data 212 of a geographical position of the wearer 1 is detected by the position unit 15. The position data 212 may, for example, provide information as to whether the wearer 1 is located in a risk area for epidemics or insects.

Further, the system 5 comprises an external apparatus 4 that can communicate environmental data 210 in the form of external data 213 to the clothing element 10. For this purpose, the clothing element 10 comprises the communication unit 16 for receiving the external data 213. The external apparatus 4 may be, for example, a mobile terminal, another clothing of another wearer 1 or a central control station such as a server. In particular, the communication unit 16 and the external apparatus 4 may be configured for bidirectional communication.

For producing 102 an evaluation 220 of a potential danger of an advance of the organic structures 2 towards the wearer 1 dependent on the environmental data 210, the control unit 20 comprises an evaluation module 22 which is in communication with the emission module 21 of the control unit 20. The emission module 21 is adapted to control the emission of the radiation 200 of the emission unit 12 in dependence on the environmental data 210, in particular in dependence on the evaluation of the environmental data 210. As a result, a generation 103 of the protected area 201 can be performed to protect the wearer 1, in particular while minimizing a use of the electromagnetic radiation 200.

FIG. 4 shows a system 5 according to the invention with a clothing element 10 according to the invention in a second embodiment. Here, the system 5 and its function essentially correspond to the system 5 and its function of the first embodiment example. However, instead of arranging the components in the shield section 17, a energy supply unit 13, a control unit 20 and preferably a position (detection) unit 15 and a communication unit 16 are arranged on the wearing portion 11, in particular in an occipital region. As a result, a weight and a space requirement of the components of the clothing element 10 can be distributed, so that a comfort of the clothing element 10 and thus its acceptability is increased. Furthermore, this arrangement offers advantages when the energy supply unit 13 comprises a connection element 13.1 for connection to an external energy source 3, such as a power socket or a USB socket, as shown in FIG. 4. If the connection element 13.1, in particular in the form of a USB interface, is designed/configured for a cable connection, the arrangement can further prevent the cable from interfering in the facial area. It is conceivable that the energy supply unit 13 additionally comprises an internal energy storage 13.2 which can be charged via the connecting element 13.1.

FIG. 5 shows a system 5 according to the invention with a clothing element 10 according to the invention in a further embodiment. In this case, the clothing element 10 is designed/configured as a pair of glasses. For this purpose, two wearing portions 11 in the form of glasses temples are provided. A control unit 20, a communication unit 16 for communicating with an external apparatus 4 and a energy supply unit 13 are integrated in the wearing portions 11. Furthermore, the clothing element 10 comprises a sensor unit 14 for capturing 101 environmental data 210, which is directed in the viewing direction of a wearer 1. On a side of the clothing element 10 opposite to the sensor unit 14, an emission unit 12 for emitting an electromagnetic radiation 200 for killing organic structures 2 is further arranged. By means of the radiation 200, a protected area 201 can be generated by which a body region 202 in the form of a face area of the wearer 1 is at least partially protected when the clothing element 10 is in a wearing position I in which the clothing element 10 is worn by the wearer 1. Since the eyes of the wearer 1 are at least partially protected by the glasses anyway, it may be sufficient if the protected area 201 only protects the nose and the mouth of the wearer 1 at least partially from the organic structures 2.

FIG. 6 shows a system 5 according to the invention with a clothing element 10 according to the invention in a further embodiment. In this case, the clothing element 10 is designed/configured essentially in accordance with the first embodiment example. However, in contrast to the first embodiment example, the clothing element 10 comprises a plurality of emission units 12 for emitting an electromagnetic radiation 200 for killing organic structures 2. Preferably, each of the emission units 12 comprises at least one emission element 12.1 or a plurality of emission elements 12.1 as described in the preceding embodiments. Further, the emission units 12 may each have different radiation parameters. For example, electromagnetic radiation 200 having a different wavelength and/or intensity may be emitted by each of the emission units 12. Depending on the environmental situation, a different emission unit 12 may therefore be controllable by a control unit 20 in order to enable radiation emission appropriate to the situation. For this purpose, the emission units 12 are further arranged in succession in such a way that a body region 202 of a wearer 1 in a wearing position I of the clothing element 10 can be at least partially or completely protected from the organic structures 2 by each of the emission units 12. As shown, the emission units 12 may be arranged for this purpose along a viewing direction of the wearer 1 at a shield section 17, for example.

In any of the previously described embodiments, it is conceivable that the clothing element 10 is designed/configured without the communication unit 16 and can thus be used independently of the external apparatus 4 to protect the wearer.

The foregoing explanation of the embodiments describes the present invention by way of example only. Of course, individual features of the embodiments can be freely combined with each other, provided that this is technically reasonable, without leaving the scope of the present invention.

### LIST OF REFERENCE SIGNS

1 Wearer
2 Structures
3 External energy source
4 Apparatus
5 System
10 Clothing element
11 Wearing portion
12 Emission unit
12.1 Emission element
13 Energy supply unit
13.1 Connecting element
13.2 Internal energy storage
14 Sensor unit
15 Position unit
16 Communication unit
17 Shield section
20 Control unit
21 Emission module
22 Evaluation module
100 Method
101 Detection from 210
102 Production from 220
103 Generation from 201
200 Radiation
200.1 Opening angle
201 Protected area
201.1 Radiation direction
202 Body region
210 Environmental data
211 Sensor data
212 Position data
213 Data
220 Evaluation
I Wearing position

1-16. (canceled)

17. A clothing element comprising
a wearing portion for wearing the clothing element in a wearing position by a wearer, and
an emission unit for emitting electromagnetic radiation for killing organic structures,
wherein, in the wearing position, a protected area can be generated by the electromagnetic radiation of the emission unit, by means of which a body region of the wearer is at least partially protected from the organic structures.

18. The clothing element according to claim 17,
wherein
the radiation emittable by the emission unit comprises a UV radiation.

19. The clothing element according to claim 17,
wherein
the emission unit has at least one emission element in the form of at least an LED, a laser or an excimer element.

20. The clothing element according to claim 17,
wherein
the electromagnetic radiation in the wearing position has a radiation direction by means of which the electromagnetic radiation is prevented from contacting on the body region.

21. The clothing element according to claim 17,
wherein
the emission unit is configured to emit the radiation in a conical manner.

22. The clothing element according to claim 17,
wherein
the body region at least partially protected by the protected area is a facial region of the wearer.

23. The clothing element according to claim 17,
wherein
the wearing portion is configured for wearing the clothing element on the head of the wearer, wherein a shield section extends from the wearing portion for shielding the body region on which the emission unit is arranged.

24. The clothing element according to claim 17,
wherein
an energy supply unit for providing electrical energy for the emission unit is provided.

25. The clothing element according to claim 17,
wherein
an internal control unit with an emission module for controlling the emission of the emission unit is provided.

26. The clothing element according to claim 17,
wherein
the control unit is connected to a sensor unit for capturing environmental data in the form of sensor data.

27. The clothing element according to claim 17,
wherein
the control unit is connected to a position unit for capturing environmental data in the form of position data of a geographical position of the clothing element.

28. The clothing element according to claim 17,
wherein
the control unit is connected to a communication unit for receiving environmental data in the form of external data from an external apparatus.

29. The clothing element according to claim 17,
wherein
the control unit comprises an evaluation module for producing an evaluation of a hazard potential of penetration of organic structures to the wearer as a function of the environmental data.

30. The clothing element according to claim 17,
wherein
a plurality of emission units are provided for emitting electromagnetic radiation for killing organic structures.

31. A system comprising
at least one clothing element, according to claim 17, and
an external apparatus for data communication with the clothing element,
wherein the clothing element has a wearing portion for wearing the clothing element in a wearing position by a wearer, and an emission unit for emitting electromagnetic radiation for killing organic structures,
wherein, in the wearing position, a protected area can be generated by the electromagnetic radiation of the emission unit, by means of which a body region of the wearer is at least partially protected from the organic structures,
wherein the clothing element comprises a communication unit for receiving environmental data in the form of external data from the external apparatus,
wherein an evaluation module is provided for producing an evaluation of a hazard potential of penetration of organic structures to the wearer in dependence on the environmental data,
wherein the clothing element comprises an internal control unit with an emission module for controlling the emission of electromagnetic radiation of the emission unit in dependence on the evaluation.

32. A method for protecting a body region of a wearer of a clothing element, according to claim 17, from organic structures while the clothing element is in a wearing position in which the clothing element is worn by the wearer, comprising:
Capturing of environmental data of an environment of the wearer,
producing an evaluation of a hazard potential of penetration of organic structures to the wearer in dependence on the environmental data,
generating a protected area in dependence on the evaluation by emitting electromagnetic radiation for killing the organic structures by the clothing element, so that a body region of the wearer is at least partially protected from the organic structures by the protected area.

33. The clothing element according to claim 24,
wherein
the energy supply unit comprising a connecting element for connection to at least an external energy source or an internal energy storage.

34. The clothing element according to claim 30,
wherein
at least the emission units having different radiation parameters or the emission units being arranged successively in such a way that the body region in the wearing position can be at least partially protected by each of the emission units.

35. The clothing element according to claim 29,
wherein
the emission module being configured for controlling the emission of the electromagnetic radiation of the emission unit depending on the evaluation.

36. The clothing element according to claim 26,
wherein
the emission module being configured for controlling the emission of the electromagnetic radiation of the emission unit as a function of the sensor data.
